(19) United States
(12) Patent Application Publication
(10) Pub. No.: US 2007/0254371 A1
(43) Pub. Date: Nov. 1, 2007

(54) Peptide combos and their uses

(75) Inventors: Koen Kas, Schilde (BE); Joel Vandekerckhove, Loppem (BE); Luc Krols, Brasschaat (BE)

Correspondence Address: TRASK BRITT, P.O. BOX 2550, SALT LAKE CITY, UT, 84110, US

(73) Assignees: Vlaams Interuniversitair Instituut Voor Biotechnologie VZW, Zwijnaarde (BE); Universiteit Gent, Gent (BE)

(21) Appl. No.: 11/305,737

(22) Filed: Dec. 16, 2005

Related U.S. Application Data
(63) Continuation of application No. PCT/EP04/51158, filed on Jun. 17, 2004.
(60) Provisional application No. 60/479,061, filed on Jun. 17, 2003.

(30) Foreign Application Priority Data
Jun. 17, 2003 (EP) 03101775.9

(51) Int. Cl. G01N 33/00; C07K 14/00; C12N 9/14

(52) U.S. Cl. 436/86; 435/195; 530/350

(57) ABSTRACT

The invention provides reagents and methods for the accurate quantification of proteins in complex biological samples. Quantification is obtained by adding to a sample a peptide combo, which is essentially a collection of synthetic reference peptides. The synthetic reference peptides have a small mass difference when compared to the biological reference peptides that originate upon digestion from the proteins present in the sample. Reference peptides and synthetic reference peptides are selected and the identity and accurate amounts of reference peptides are determined by mass spectrometry. The methods can be used in high throughput assays to interrogate proteomes.

### CROSS-REFERENCE TO RELATED APPLICATIONS

This application is a continuation of PCT International Patent Application No. PCT/EP2004/051158, filed on Jun. 17, 2004, designating the United States of America, and published, in English, as PCT International Publication No. WO 2004/111636 A2 on Dec. 23, 2004, which application claims priority to U.S. Provisional Patent Application Ser. No. 60/479,061, filed Jun. 17, 2003, and European Patent Application Serial No. 03101775.9, also filed Jun. 17, 2003, the contents of the entirety of each of which are hereby incorporated by this reference.

### STATEMENT ACCORDING TO 37 C.F.R. § 1.52(e)(5)—SEQUENCE LISTING SUBMITTED ON COMPACT DISC

A sequence listing will be submitted at a later time.

### STATEMENT ACCORDING TO 37 C.F.R. § 1.52(e)(5)—TABLES SUBMITTED ON COMPACT DISC

Pursuant to 37 C.F.R. § 1.52(e)(1)(iii), a compact disc containing an electronic version of all of the tables in the application has been submitted concomitant with this application, the contents of which are hereby incorporated by reference. A second compact disc is submitted and is an identical copy of the first compact disc. The discs are labeled “copy 1” and “copy 2,” respectively, and each disc contains one file entitled “Tables.doc” which is 13,605 KB and created on Dec. 16, 2005.

### TECHNICAL FIELD

The invention provides reagents and methods for the accurate quantification of proteins in complex biological samples. Quantification is obtained by adding to a sample a peptide combo which is essentially a collection of synthetic reference peptides. The synthetic reference peptides have a small mass difference when compared to the biological reference peptides that originate upon digestion from the proteins present in the sample. Reference peptides and synthetic reference peptides are selected and the identity and accurate amounts of reference peptides are determined by mass spectrometry. The methods can be used in high throughput assays to interrogate proteomes.

### BACKGROUND

Proteomics comprises the large-scale study of protein expression, protein interactions, protein function and protein structure. For years, the method to determine the proteome in a target tissue or cells has been two-dimensional polyacrylamide gel electrophoresis (2D-PAGE). 2D-PAGE produces separations of proteins in complex mixtures, based on their difference in size (molecular weight) and isoelectric point (pI) and displays protein spots in a 2D pattern. 2D-PAGE is sequential, labor intensive, and difficult to automate. Furthermore, specific classes of proteins, such as membrane proteins, very large and small proteins, and highly acidic or basic proteins, are difficult to analyze using this method. Because of such shortcomings, gel-free systems have been developed, in which proteins are identified based on the mass of one or more of their constituting peptides, without first separating the individual proteins on a gel.

One approach is the Multidimensional protein identification technology (MudPIT) (Washburn et al., Nat. Biotech. 19, 242-247, 2001). MudPIT separates a complex peptide mixture via a cation exchange (separation on charge) followed by a reverse phase chromatography (separation on hydrophobicity). Following digestion all peptides are analyzed, none are pre-sorted.

A second approach is a methodology that makes use of a chemical labeling reagent called ICAT (Isotope Coded Affinity Tag, Applied Biosystems) (Gygi et al., Nat. Biotech. 17, 994-999, 1999). This ICAT method is based on the specific binding of an iodoacetate derivative carrying a biotin label to peptides containing a cysteine residue (Cys-peptides). The samples are mixed, and enzymatically digested. The peptide mixture is run over an affinity purification column with streptavidine beads, and only the Cys-peptides are retained on the column. The Cys-peptides are subsequently eluted and analyzed with a mass spectrometer.

A third approach, designated as COFRADIC (combined fractional diagonal chromatography, described in WO02077016) is also a gel-free methodology but this technology does not use affinity tags for its selection of peptides. The basic strategy of COFRADIC comprises a combination of two chromatographic separations of the same type, separated by a step in which the selected population of peptides is altered in such a way that the chromatographic behavior of the altered peptides in the second chromatographic separation differs from the chromatographic behavior of its unaltered version. COFRADIC and comparable technologies allows exploration of the profile of large sets of proteins in two or more samples.

For many applications however, it would be advantageous to be able to focus on the profile of a limited number of proteins. Traditionally, antibody-based approaches (ELISA, Western, antibody-based protein chips) have been used to explore the expression patterns of proteins. A disadvantage of these approaches is the time-consuming step to raise and characterize antibodies against each of the target proteins to be analyzed. Also, an antibody that binds a native protein (as in immuno precipitation) may not be useful for detecting the denatured protein on a Western blot. Thus, a technique that yields results similar to the antibody based approaches but does not require antibodies could have significant advantages. Indeed, WO03/016861 and WO02/084250 describe the detection and quantification of target proteins in biological samples through the use of a synthetic labeled reference peptide. In a mass spectrum the synthetic labeled reference peptide appears as a doublet with the peptide derived from the target peptide. A comparison of the peak highs is used for accurate quantification of the target protein. However, these methods do not use a pre-sorting of the target peptides which results in an overwhelming of the resolution power of any known chromatography system. In addition, the resolving power of MS coupled with such chromatography is not sufficient to adequately determine the mass of a representative number of individual target peptides. Thus, there is a need for an alternative methodology capable of accurate quantification of one or more specific proteins out of extremely complex mixtures without bias or need for extensive purification of intact proteins.

### SUMMARY OF THE INVENTION

In the present invention, we have used a combination of synthetic peptides (herein further called a peptide combo) and the COFRADIC technology and we have surprisingly found that proteins of interest can be detected and quantified in a complex mixture with great sensitivity, dynamic range, precision and speed. In our methodology, quantification is obtained by adding to a sample a known amount of synthetic reference peptides. The power of using the COFRADIC technology is that it is capable of specifically selecting for these synthetic reference peptides together with the natural reference peptides in the second chromatographic step. An advantage of our invention is that it is an extremely flexible technology since it can select for reference peptides specifically altered on an amino acid of interest, such as, for example, methionine, cysteine, a combination of methionine and cysteine, amino-terminal peptides, phosphorylated peptides and acetylated peptides.

In the present invention, peptide combos allow to quickly interrogate complex protein mixtures and to perform absolute protein quantification. In principle, peptide Combos can be designed for any set of target proteins. A set of target proteins is, for instance, the family of G-protein coupled receptors or the tyrosine kinases, or the proteins involved in a particular signal transduction pathway. To our knowledge, there are no comparable, equally versatile technologies available to rapidly evaluate specific sets of proteins. For instance, in the case of membrane proteins, many of the issues surrounding protein solubility are avoided since a soluble proteolytic peptide may be chosen to represent the intact protein. The present invention can be developed for rapid and sensitive, quantitative biomarker studies (prognosis, diagnosis, and therapy monitoring in large populations), as well as for drug target validation and pathway analysis.

### BRIEF DESCRIPTION OF THE DRAWING

FIG. 1: Seven different isoforms of VEGF-A (VEGF-A_{—}206, VEGF-A_{—}189, VEGF-A_{—}183, VEGF-A_{—}165, VEGF-A_{—}148, VEGF-A_{—}145, VEGF-A_{—}121) with the position of CYS-containing peptides indicated. No peptides can be defined for VEGF-A_{—}165 and VEGF-A_{—}148.

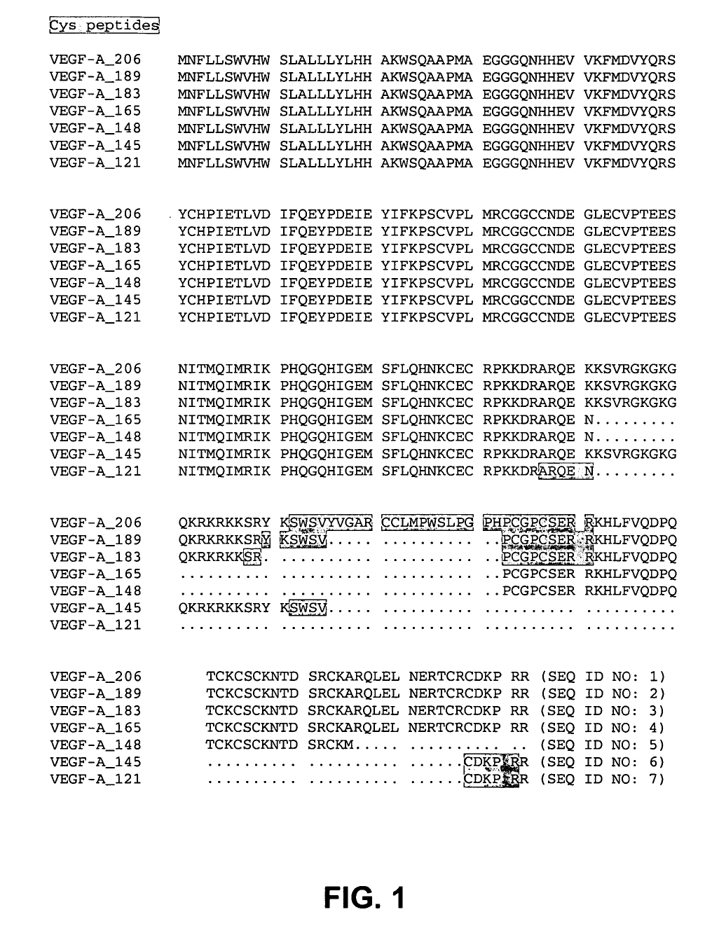

### DETAILED DESCRIPTION OF THE INVENTION

The following definitions are provided for specific terms which are used in the written description.

As used in the specification and claims, the singular form “a,” “an” and “the” include plural references unless the context clearly dictates otherwise. For example, the term “a cell” includes a plurality of cells, including mixtures thereof. The term “a protein” includes a plurality of proteins.

“Protein,” as used herein, means any protein, including, but not limited to peptides, enzymes, glycoproteins, hormones, receptors, antigens, antibodies, growth factors, etc., without limitation. Presently preferred proteins include those comprised of at least 25 amino acid residues, more preferably, at least 35 amino acid residues and still more preferably, at least 50 amino acid residues. The terms “polypeptide” and “protein” are generally used interchangeably herein to refer to a polymer of amino acid residues.

As used herein, the term “peptide” refers to a compound of two or more subunit amino acids. The subunits are linked by peptide bonds.

As used herein, a “target protein” or a “target polypeptide” is a protein or polypeptide whose presence or amount is being determined in a protein sample by use of one or more synthetic reference peptides. In a preferred embodiment, it is understood that the target peptide or target protein belongs to a family of proteins. The target protein/polypeptide may be a known protein (i.e., previously isolated and purified) or a putative protein (i.e., predicted to exist on the basis of an open reading frame in a nucleic acid sequence). For each target protein at least one synthetic reference peptide is chosen and synthesized. Such open reading frames can be identified from a database of sequences including, but not limited to, the GenBank database, EMBL data library, the Protein Sequence Database and PIR International, SWISS-PROT, The ExPASy proteomics server of the Swiss Institute of Bioinformatics (SIB) and databases described in PCT/US01/25884. Predicted cleavage sites also can be identified through modeling software, such as IVIS-Digest (available at http://prospector.ucsf.edu/). Predicted sites of protein modification also can be determined using software packages, such as Scansite, Findmod, NetOGlyc (for prediction of type-O-glycosylation sequences), YinOYang (for prediction of O-beta-GlcNac attachment sites), big-PI Predictor (for prediction of GPI modifications), NetPhos (for prediction of Ser, Thr, and Tyr phosphorylation sites), NMT (for prediction of N-terminal N-myristolation) and Sulfinator (for prediction of tyrosine sulfation sites) which are accessible through ˜, for example. A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Preferably, therefore, a peptide is at least about four amino acids. The size of the peptide is also optimized to maximize ionization frequency. As used herein, a “protease activity” is an activity, which cleaves amide bonds in a protein or polypeptide. The activity may be implemented by an enzyme, such as a protease or by a chemical agent, such as CNBr.

As used herein, “a protease cleavage site” is an amide bond, which is broken by the action of a protease activity.

As used herein, a “labeled reference peptide” is a labeled peptide internal standard and refers to a synthetic peptide which corresponds in sequence to the amino acid subsequence of a known protein or a putative protein predicted to exist on the basis of an open reading frame in a nucleic acid sequence and which is preferentially labeled by a mass-altering label, such as a stable isotope. The boundaries of a labeled reference peptide are governed by protease cleavage sites in the protein (e.g., sites of protease digestion or sites of cleavage by a chemical agent, such as CNBr). Protease cleavage sites may be predicted cleavage sites (determined based on the primary amino acid sequence of a protein and/or on the presence or absence of predicted protein modifications, using a software modeling program) or may be empirically determined (e.g., by digesting a protein and sequencing peptide fragments of the protein).

As used herein, a “cell state profile” or a “tissue state profile” refers to values of measurements of levels of one or more proteins in a cell or tissue. Preferably, such values are obtained by determining the amount of peptides in a sample having the same peptide fragmentation signatures as that of peptide internal standards corresponding to the one or more proteins. A “diagnostic profile” refers to values that are diagnostic of a particular cell state, such that when substantially the same values are observed in a cell, that cell may be determined to have the cell state. For example, in one aspect, a cell state profile comprises the value of a measurement of p53 expression in a cell. A diagnostic profile would be a value which is significantly higher than the value determined for a normal cell and such a profile would be diagnostic of a tumor cell.

The term “sample” generally refers to a “biological sample” and comprises any material directly or indirectly derived from any living source (e.g., plant, human, animal, microorganism, such as fungi, bacteria, virus). Examples of appropriate biological samples for use in the invention include: tissue homogenates (e.g., biopsies), cell homogenates; cell fractions; biological fluids (e.g., urine, serum, cerebrospinal fluid, blood, saliva, amniotic fluid, mouth wash); and mixtures of biological molecules including proteins, DNA, and metabolites. The term also includes products of biological origin including pharmaceuticals, nutraceuticals, cosmetics, and blood coagulation factors, or the portion(s) thereof that are of biological origin e.g., obtained from a plant, animal or microorganism. Any source of protein in a purified or non-purified form can be utilized as starting material, provided it contains or is suspected of containing the protein of interest. Thus, the target protein of interest may be obtained from any source, which can be present in a heterogeneous biological sample. The sample can come from a variety of sources. For example: 1) in agricultural testing the sample can be a plant, plant-pathogen, soil residue, fertilizer, liquid or other agricultural product; 2) in food testing the sample can be fresh food or processed food (for example, infant formula, fresh produce, and packaged food); 3) in environmental testing the sample can be liquid, soil, sewage treatment, sludge, and any other sample in the environment which is required for analysis of a particular protein target; 4) in pharmaceutical and clinical testing the sample can be animal or human tissue, blood, urine, and infectious diseases.

Proteomics is the systematic identification and characterization of proteins for their structure, function, activity, quantity, and molecular interaction. In quantitative proteomics information is sought about accurate protein expression levels. Methods for absolute quantification are described in the art whereby synthetic peptides comprising stable isotopes are used. The present invention provides an alternative method for the quantitative determination of target proteins in one or more samples. The invention is based on a selection (sorting) of only a subset of peptides out of a sample comprising a protein peptide mixture and a peptide combo (a set of synthetic reference peptides). The peptide combo is specifically designed such that its synthetic reference peptides can be captured (sorted) in the COFRADIC selection process.

The present invention is more flexible than existing methods because the selection of peptides can be adapted according to the scientist's choice since different amino acids present in the reference peptides can be used for sorting. Or, in other words, a reference peptide can be selected that comprises an amino acid that can be specifically altered. The target protein, preferentially belonging to a family of proteins, can be digested e.g., cleaved by a specific protease, to generate a family of peptide fragments that can be analyzed by mass spectrometry to generate a peptide mass fingerprint. As used herein the term “signature peptide masses” refers to the peptide masses generated from a particular protein target or targets, which can used to identify the protein target. Those peptide masses from a given peptide mass fingerprint which ionize easily and have a high mass resolution and accuracy, are considered to be members of a set of signature diagnostic peptide masses for a given target. The pattern is unique and, thus, distinct for each protein.

One skilled in the art will recognize that peptide mass fingerprints generated from a protein target can be compared with predicted peptide mass fingerprints generated in silico and predicted masses of a target protein. Thus, the location of where these peptide masses reside in a given target protein can be determined (e.g., a peptide fragment may reside near the N-terminus or C-terminus of a protein). The observed peptide masses of a target protein can be compared with in silico predicted masses of a target protein for which the amino acid sequence is known. Those peptide masses from a given peptide mass fingerprint, which ionize easily and have high mass resolution and accuracy are considered to be members of a signature diagnostic peptide mass for a given target. Once a set of signature diagnostic peptide masses have been identified from a protein target, it is possible to detect or determine the absolute amount of the target protein in a complex mixture by using synthetic reference peptides. For quantification, a known amount of synthetic reference peptides (which serve as internal standards), at least one such peptide and in preferred embodiments, two for each specific protein in the mixture to be detected or quantified, are added to the sample to be analyzed. Quantification of target proteins in one or more different samples containing protein mixtures (e.g., biological fluids, cell or tissue lysates, etc.) can be determined using synthetic reference peptides based upon in silico proteolytic digests of targeted proteins, which have been modified as to change the mass. The amounts of a given target protein in each sample is determined by comparing the abundance of the mass-modified reference peptides from any modified peptide originating from that protein. The method can be used to quantify amounts of known proteins in different samples. It is thus possible to determine the absolute amounts of specific proteins in a complex mixture. In this case, a known amount of a synthetic reference peptide, at least one for each specific protein in the mixture to be quantified is added to the sample to be analyzed. Accurate quantification of the target protein is achieved through the use of synthetically modified reference peptides that have amino acid identity, or near identity, to signature diagnostic peptides and has been predetermined for molecular weight and mass. The typical quantification analysis is based on two or more signature diagnostic peptides that are measured to reduce statistical variation, provide internal checks for experimental errors, and provide for detection of post-translation modifications.

The method of this invention can be used for quantitative analysis of single or multiple target proteins in complex biological samples for a variety of applications that include agricultural, food monitoring, pharmaceutical, clinical, production monitoring, quality assurance and quality control, and the analysis of environmental samples.

In the present invention, a reference peptide is a peptide that allows unambiguous identification of its parent protein. Thus, every target protein to be quantified should be represented by at least one and, preferably, two or more reference peptides. A reference peptide can be an amino-terminal peptide, or a carboxy-terminal peptide but can also be an internal peptide derived from a protein. The quantification is obtained by adding a known amount of the synthetic counterpart of the reference peptide, whereby the reference peptide differs from its synthetic counterpart by a differential isotopic labeling which is sufficiently large to distinguish both forms in conventional mass spectrometers.

In one embodiment, the invention provides a process to identify a peptide combo wherein the peptide combo corresponds with a family of proteins and wherein each of the members of the peptide combo is derived from an unique protein from the family comprising (a) generating peptides by applying an in silico digest on the family of proteins, (b) constructing a relational database comprising the peptides with a predicted mono-isotopic weight within the range of 400 to 5000 Da, and (c) identifying a peptide combo with chosen properties.

A peptide combo in the present invention is defined as a collection of at least two synthetic reference peptides. Preferentially, a peptide combo corresponds to a family of proteins. With the wording “a family of proteins” it is meant a group of proteins that are functionally linked together because the proteins are in the same pathway (a MAP-kinase pathway, a hedgehog pathway, an apoptotic process), or the proteins have a role in the same pathology (e.g., a neurodegenerative process, Alzheimer's disease, psoriasis), or the proteins are substrates for the same protease (e.g., gamma-secretase, a matrix metalloproteinase), or the proteins have the same function (kinases, glycosylating enzymes), or the proteins have a similar structure (e.g., G-protein coupled receptors) or the proteins have the same subcellular localization (e.g., post-synaptic vesicles, endoplasmic reticulum). The wording “in silico” digest is clarified herein further.

Since the invention provides (labeled) synthetic reference peptides as internal standards for use in determining the presence of, and/or quantifying the amount of, at least one target protein in a sample which comprises an amino acid subsequence identical to the peptide portion of the internal standard. Reference peptides are generated by examining the primary amino acid sequence of a protein and synthesizing a peptide comprising the same sequence as an amino acid subsequence of the protein. In one aspect, the peptide's boundaries are determined by “in silico” predicting the cleavage sites of a protease. In another aspect, a protein is digested by the protease and the actual sequence of one or more peptide fragments is determined. Suitable proteases include, but are not limited to one or more of: serine proteases (e.g., such as trypsin, pepsin, SCCE, TADG12, TADG14); metallo-proteases (e.g., such as PUMP-1); chymotrypsin; cathepsin; pepsin; elastase; pronase; Arg-C; Asp-N; Glu-C; Lys-C; carboxypeptidases A, B, and/or C; dispase; thermolysin; cysteine proteases such as gingipains, and the like. Proteases may be isolated from cells or obtained through recombinant techniques. Chemical agents with a protease activity also can be used (e.g., such as CNBr).

A “relational database” means a database in which different tables and categories of the database are related to one another through at least one common attribute and is used for organizing and retrieving data. The term “external database” as used herein refers to publicly available databases that are not a relational part of the internal database, such as GenBank and Blocks.

A “predicted mono-isotopic weight within the range of 400 to 5000 Da” means that the peptides are preferentially larger than four amino acids and smaller than 50 amino acids. More preferably, the mono-isotopic weight is within the range of 500 to 4500 Da and even more preferably, the weight is within the range of 600 to 4000 Da.

The peptide combo is designed such that the reference peptides of the peptide combo can identify the family of proteins of interest. In a preferred embodiment, the peptide combo is a representative of more than 90%, preferentially more than 95% and even more preferentially 100% of the family of proteins.

In a particular embodiment, the family of proteins are membrane proteins and the peptides in the relational database have less than 20% coverage in the transmembrane area. In a more particular embodiment, the peptides have less than 15%, 10%, 5% or even less coverage in the transmembrane area. In another particular embodiment, the transmembrane proteins are G-protein coupled receptors.

In a particular embodiment, the invention provides a peptide combo that comprises at least two synthetic reference peptides. Preferably, the peptide combo comprises at least 3, 4, 5, 6, 7, 8, 9, 10, 15, 20, 25, 30, 35, 40, 45, 50, 55, 60, 65, 70, 75, 80, 85, 90, 95, 100 or even more synthetic reference peptides.

In another particular embodiment, the reference peptides are isotopically labeled. In yet another particular embodiment, the reference peptides are derived from G-protein coupled receptors. In yet another embodiment, the reference peptides are derived from protease substrates. In yet another embodiment, the protease substrates are generated by gamma secretase.

The synthetic reference peptides of the present invention (the peptide combos) are herein used in combination with the gel-free proteomics technology designated as COFRADIC. The COFRADIC technology is fully described in WO02077016, which is herein incorporated by reference. However, to clarify the COFRADIC concept, the most important elements are herein repeated. Essentially, COFRADIC utilizes a combination of two chromatographic separations of the same type, separated by a step in which a selected population of the peptides is altered in such a way that the chromatographic behavior of the altered peptides in the second chromatographic separation differs from the chromatographic behavior of its unaltered version. To isolate a subset of peptides out of a protein peptide mixture, COFRADIC can be applied in two action modes. In a first mode, a minority of the peptides in the protein peptide mixture is altered and the subset of altered peptides is isolated.

In a second, reverse mode, the majority of the peptides in the protein peptide mixture are altered and the subset of unaltered peptides is isolated. The same type of chromatography means that the type of chromatography is the same in both the initial separation and the second separation. The type of chromatography is, for instance, in both separations based on the hydrophobicity of the peptides. Similarly, the type of chromatography can be based in both steps on the charge of the peptides and the use of ion-exchange chromatography.

In still another alternative, the chromatographic separation is in both steps based on a size exclusion chromatography or any other type of chromatography. The first chromatographic separation, before the alteration, is hereinafter referred to as the “primary run” or the “primary chromatographic step” or the “primary chromatographic separation” or “run 1.” The second chromatographic separation of the altered fractions is hereinafter referred to as the “secondary run” or the “secondary chromatographic step” or the “secondary chromatographic separation” or “run 2.”

In a preferred embodiment of the invention, the chromatographic conditions of the primary run and the secondary run are identical or, for a person skilled in the art, substantially similar. “Substantially similar” means, for instance, that small changes in flow and/or gradient and/or temperature and/or pressure and/or chromatographic beads and/or solvent composition is tolerated between run 1 and run 2 as long as the chromatographic conditions lead to an elution of the altered peptides that is predictably distinct from the non-altered peptides and this for every fraction collected from run 1. As used herein, a “protein peptide mixture” is typically a complex mixture of peptides obtained as a result of the cleavage of a sample comprising proteins. Such sample is typically any complex mixture of proteins, such as, without limitation, a prokaryotic or eukaryotic cell lysate or any complex mixture of proteins isolated from a cell or a specific organelle fraction, a biopsy, laser-capture dissected cells or any large protein complexes, such as ribosomes, viruses and the like. It can be expected that when such protein samples are cleaved into peptides that they may contain easily up to 1,000, 5,000, 10,000, 20,000, 30,000, 100,000 or more different peptides. However, in a particular case a “protein peptide mixture” can also originate directly from a body fluid or more generally any solution of biological origin. It is well known that, for example, urine contains, besides proteins, a very complex peptide mixture resulting from proteolytic degradation of proteins in the body of which the peptides are eliminated via the kidneys.

Yet another illustration of a protein peptide mixture is the mixture of peptides present in the cerebrospinal fluid. The term “altering” or “altered” or “alteration” as used herein in relation to a peptide, refers to the introduction of a specific modification in an amino acid of a peptide, with the clear intention to change the chromatographic behavior of such peptide containing the modified amino acid. An “altered peptide” as used herein is a peptide containing an amino acid that is modified as a consequence of an alteration. Such alteration can be a stable chemical or enzymatical modification. Such alteration can also introduce a transient interaction with an amino acid. Typically, an alteration will be a covalent reaction, however, an alteration may also consist of a complex formation, provided the complex is sufficiently stable during the chromatographic steps. Typically, an alteration results in a change in hydrophobicity such that the altered peptide migrates different from its unaltered version in hydrophobicity chromatography. Alternatively, an alteration results in a change in the net charge of a peptide, such that the altered peptide migrates different from its unaltered version in an ion exchange chromatography, such as an anion exchange or a cation exchange chromatography. Also, an alteration may result in any other biochemical, chemical or biophysical change in a peptide such that the altered peptide migrates different from its unaltered version in a chromatographic separation. The term “migrates differently” means that a particular altered peptide elutes at a different elution time with respect to the elution time of the same non-altered peptide. Altering can be obtained via a chemical reaction or an enzymatic reaction or a combination of a chemical and an enzymatic reaction.

A non-limiting list of chemical reactions includes alkylation, acetylation, nitrosylation, oxidation, hydroxylation, methylation, reduction and the like. A non-limiting list of enzymatic reactions includes treating peptides with phosphatases, acetylases, glycosidases or other enzymes which modify co- or post-translational modifications present on peptides. The chemical alteration can comprise one chemical reaction, but can also comprise more than one reaction (e.g., a β-elimination reaction and an oxidation), such as, for instance, two consecutive reactions in order to increase the alteration efficiency. Similarly, the enzymatic alteration can comprise one or more enzymatic reactions.

Another essential feature of the alteration in the current invention is that the alteration allows the isolation of a subset of peptides out of a protein peptide mixture. A chemical and/or enzymatic reaction which results in a general modification of all peptides in a protein peptide mixture will not allow the isolation of a subset of peptides. Therefore, an alteration has to alter a specific population of peptides in a protein peptide mixture to allow for the isolation of a subset of peptides in the event such alteration is applied in between two chromatographic separations of the same type.

In a preferred embodiment, the specific amino acid selected for alteration comprises one of the following amino acids: methionine (Met), cysteine (Cys), histidine (His), tyrosine (Tyr), lysine (Lys), tryptophan (Trp), arginine (Arg), proline (Pro) or phenylalanine (Phe). Importantly is that the alteration can also be specifically targeted to a population of amino acids carrying a co- or posttranslational modification. Examples of such co- or posttranslational modifications are glycosylation, phosphorylation, acetylation, formylation, ubiquitination, pyrroglutamylation, hydroxylation, nitrosylation, ε-N-acetylation, sulfation, NH₂-terminal blockage. Examples of modified amino acids altered to isolate a subset of peptides according to the current invention are phosphoserine (phospho-Ser), phospho-threonine (phospho-Thr), phospho-histidine (phosho-His), phospho-aspartate (phospho-Asp) or acetyl-lysine.

A further non-limiting list of examples of amino acids that can be altered and can be used to select a subset of peptides are other modified amino acids (e.g., a glycosylated amino acid), artificially incorporated D-amino acids, seleno-amino acids, amino acids carrying an unnatural isotope and the like. An alteration can also target a particular residue (e.g., a free NH₂-terminal group) on one or more amino acids or modifications added in vitro to certain amino acids. Alternatively the specific chemical and/or enzymatic reaction has a specificity for more than one amino acid residue (e.g., both phosphoserine and phosphothreonine or the combination of methionine and cysteine) and allows separation of a subset of peptides out of a protein peptide mixture. Typically, the number of selected amino acids to be altered will however be one, two or three.

In another aspect, two different types of selected amino acids can be altered in a protein peptide mixture and a subset of altered peptides containing one or both altered amino acids can be isolated.

In yet another aspect, the same peptide mixture can be altered first on one amino acid, a subset of altered peptides can be isolated and, subsequently, a second alteration can be made on the remaining previously unaltered sample and another subset of altered peptides can be isolated. Thus, “reference peptides” as used herein are peptides whose sequence and/or mass is sufficient to unambiguously identify its parent protein.

By preference, peptide synthesis of equivalents of reference peptides is easy. For the sake of clarity, a reference peptide as used herein is the native peptide as observed in the protein it represents, while a synthetic reference peptide as used herein is a synthetic counterpart of the same peptide. Such synthetic reference peptide is conveniently produced via peptide synthesis but can also be produced recombinantly. Peptide synthesis can, for instance, be performed with a multiple peptide synthesizer.

Recombinant production can be obtained with a multitude of vectors and hosts as widely available in the art. Reference peptides by preference ionize well in mass spectrometry. A non-limiting example of a well ionizing reference peptide is a reference peptide which contains an arginine. By preference, a reference peptide is also easy to isolate as altered peptide or as an unaltered peptide.

In the latter preferred embodiment, the reference peptide is simultaneously also an altered peptide or an unaltered peptide. A reference peptide and its synthetic reference peptide counterpart are chemically very similar, separate chromatographically in the same manner and also ionize in the same way. The reference peptide and its synthetic reference peptide counterpart are however differentially isotopically labeled. In consequence, in a preferred embodiment, whereby the reference peptide is also an altered or unaltered peptide, the reference peptide and its synthetic reference peptide counterpart are altered in a similar way and are isolated in the same fraction of the primary and the secondary run and in an eventual ternary run. However, when a reference peptide and its synthetic reference peptide are fed into an analyzer, such as a mass spectrometer, they will segregate into the light and heavy peptide. The heavy peptide has a slightly higher mass due to the higher weight of the incorporated chosen heavy isotope. Because of this very small difference in mass between a reference peptide and its synthetic reference peptide, both peptides will appear as a recognizable closely spaced twin peak in a mass spectrometric analysis. The ratio between the peak heights or peak intensities can be calculated and these determine the ratio between the amount of reference peptide versus the amount of synthetic reference peptide. Since a known absolute amount of synthetic reference peptide is added to the protein peptide mixture, the amount of reference peptide can be easily calculated and the amount of the corresponding protein in the sample comprising proteins can be calculated.

Thus, by using the COFRADIC technology an example of a protocol to determine the quantity of one target protein in a particular protein sample is as follows: (1) selection of a reference peptide from a target protein (e.g., a reference peptide comprising methionine), (2) the corresponding synthetic counterpart is chemically synthesized (e.g., as an ¹⁸O labeled product), (3) the protein sample is digested (e.g., with trypsin in H₂¹⁶O water), (4) a known amount of synthetic reference peptide is added to the resulting protein peptide mixture, (5) the mixture is subjected to the COFRADIC methodology to separate the peptides (e.g., altered on peptides comprising methionine), (6) the sorted peptides are analyzed (e.g., altered methionine-peptides are analyzed by MALDI-TOF-MS), (7) the altered reference peptide and altered synthetic reference peptide co-elute in the process and appear as twin peaks in the mass spectrum, (8) the peak surface of each of the twin peaks is calculated, (9) the ratio between both peaks allows to calculate the amount of reference peptide and, correspondingly, the amount of target protein in the particular sample. It should be clear that step (4) can be executed before step (3); that is, the synthetic reference peptide is added and the protein sample is then digested.

Importantly, the method of using a synthetic reference peptide to determine the quantity of a protein in a sample can in principle easily be expanded to determine the quantity of multiple (even more than 100) targets in a sample and, thus, measure the expression levels of many target proteins in a given sample. Obviously, this approach can also be used to measure and compare the amount of target proteins in a large number of samples. For every protein to be quantified, there is a need for at least one and, preferably, two or more reference peptides. In a particular embodiment, each synthetic reference peptides is added in an amount equimolar to the expected amount of its reference peptide counterpart.

Labeling Methods of Synthetic Reference Peptides and/or Biological Reference Peptides

In one embodiment, a peptide combo is synthesized using one or more labeled amino acids (i.e., the label is actually part of the peptides) or less preferably, labels may be attached after synthesis. By providing the label as part of the peptides, there are minimal differences in the chemical structure of a peptide internal standard and the native peptides obtained from the digestion of the target proteins with a protease activity. Preferably, the label is a mass-altering label. The type of label selected is generally based on the following considerations: The mass of the label should, preferably, be unique to shift fragment masses produced by MS analysis to regions of the spectrum with low background. The ion mass signature component is the portion of the labeling moiety which, preferably, exhibits a unique ion mass signature in mass spectrometric analyses. The sum of the masses of the constituent atoms of the label is, preferably, uniquely different than the fragments of all the possible amino acids. As a result, the labeled amino acids and reference peptides are readily distinguished from unlabeled amino acids and reference peptides by their ion/mass pattern in the resulting mass spectrum. The label should be robust under the fragmentation conditions of MS and not undergo unfavorable fragmentation.

Labeling chemistry should be efficient under a range of conditions, particularly denaturing conditions and the labeled tag, preferably, remains soluble in the MS buffer system of choice. Preferably, the label does not suppress the ionization efficiency of the protein. More preferably, the label does not alter the ionization efficiency of the protein and is not otherwise chemically reactive.

There are several methods known in the art to differentially isotopically label a reference peptide and its synthetic reference peptide. In a first approach, the reference peptide carries the uncommon isotope and the synthetic counterpart carries the natural isotope. In this approach the synthetic reference peptides can be efficiently chemically synthesized with their natural isotopes in large-scale preparations.

To label the reference peptide with an uncommon isotope, several methods to differentially isotopically label a peptide with an uncommon isotope can be applied (in vivo labeling, enzymatic labeling, chemical labeling, etc.). The isotopic labeling of a (biological) sample comprising proteins can be done in many different ways available in the art. A key element is that a particular synthetic reference peptide and its corresponding reference peptide present in the sample are identical, except for the presence of a different isotope in one or more amino acids between the synthetic reference and its corresponding counterpart.

In a typical embodiment, the isotope in the reference peptide is the natural isotope, referring to the isotope that is predominantly present in nature, and the isotope in the synthetic reference peptide is a less common isotope, hereinafter referred to as an uncommon isotope. Examples of pairs of natural and uncommon isotopes are H and D, ¹⁶O and ¹⁸O, ¹²C and ¹³C, ¹⁴N and ¹⁵N. Reference peptides labeled with the heaviest isotope of an isotopic pair are herein also referred to as heavy reference peptides. Reference peptides labeled with the lightest isotope of an isotope pair are herein also referred to as light reference peptides. For instance, a reference peptide labeled with H is called the light reference peptide, while the same reference peptide labeled with D is called the heavy reference peptide.

Reference peptides labeled with a natural isotope and its counterparts labeled with an uncommon isotope are chemically very similar, separate chromatographically in the same manner and also ionize in the same way. However, when the reference peptides are fed into an analyzer, such as a mass spectrometer, they will segregate into the light and the heavy reference peptide. The heavy reference peptide has a slightly higher mass due to the higher weight of the incorporated, chosen isotopic label. Because of the minor difference between the masses of the differentially isotopically labeled reference peptides the results of the mass spectrometric analysis of isolated altered or unaltered reference peptides will be a plurality of pairs of closely spaced twin peaks, each twin peak representing a heavy and a light reference peptide.

In one embodiment, each of the heavy reference peptides originate from the sample labeled with the heavy isotope; each of the light synthetic reference peptides present in a peptide combo originate from a chemical synthesis were the light isotope is used for synthesis.

In another embodiment, the reverse is true and each of the heavy synthetic reference peptides present in a peptide combo originate from a chemical synthesis were the heavy isotope is used for synthesis; each of the light reference peptides originate from the sample labeled with the light isotope.

Incorporation of the natural and/or uncommon isotope in reference peptides or synthetic reference peptides can be obtained in multiple ways. In one approach proteins are labeled in the cells. Cells for a first sample are, for instance, grown in media supplemented with an amino acid containing the natural isotope and cells for a second sample are grown in media supplemented with an amino acid containing the uncommon isotope.

In one embodiment, the differentially isotopically labeled amino acid is the amino acid that is selected to become altered. For instance, if methionine is the selected amino acid, cells are grown in media supplemented either with unlabeled L-methionine (first sample) or with L-methionine which is deuterated on the Cβ and Cγ position and which is, therefore, heavier by four amus. Alternatively, synthetic reference peptides could also contain deuterated arginine H₂NC—(NH)—NH—(CD₂)₃—CD—(NH₂)—COOH) which would add seven amus to the total peptide mass. It should be clear to one of skill in the art that every amino acid of which deuterated or ¹⁵N or ¹³C forms exist can be considered in this protocol. Incorporation of isotopes can also be obtained by an enzymatic approach. For instance, labeling can be carried out by treating a sample comprising proteins with trypsin in “heavy” water (H₂¹⁸O). As used herein “heavy water” refers to a water molecule in which the O-atom is the ¹⁸O-isotope.

Trypsin shows the well-known property of incorporating two oxygens of water at the COOH-termini of the newly generated sites. Thus, a sample, which has been trypsinized in H₂¹⁶O, peptides have “normal” masses, while a sample digested in “heavy water” have a mass increase of four amus corresponding with the incorporation of two ¹⁸O atoms. This difference of four amus is sufficient to distinguish the heavy and light version of the altered peptides or unaltered peptides in a mass spectrometer and to accurately measure the ratios of the light versus the heavy peptides and, thus, to determine the accurate amount of the corresponding protein in a sample.

Incorporation of the differential isotopes can further be obtained with multiple labeling procedures based on known chemical reactions that can be carried out at the protein or the peptide level. For example, proteins can be changed by the guadinylation reaction with O-methylisourea, converting NH₂-groups into guanidinium groups, thus generating homoarginine at each previous lysine position. The latter reagent can carry an uncommon isotope.

Peptides can also be changed by Shiff's-base formation with deuterated acetaldehyde followed by reduction with normal or deuterated sodiumborohydride. This reaction, which is known to proceed in mild conditions, may lead to the incorporation of a predictable number of deuterium atoms. Peptides will be changed either at the α-NH₂-group, or ε-NH₂ groups of lysines or on both. Similar changes may be carried out with deuterated formaldehyde followed by reduction with deuterated NaBD₄, which will generate a methylated form of the amino groups. The reaction with formaldehyde could be carried out either on the total protein, incorporating deuterium only at lysine side chains or on the peptide mixture, where both the α-NH₂ and lysine-derived NH₂-groups will be labeled. Since arginine is not reacting, this also provides a method to distinguish between Arg- and Lys-containing peptides. Primary amino groups are easily acylated with, for example, acetyl N-hydroxysuccinimide (ANHS). Thus, a sample can be acetylated with, for example, ¹³CH₃CO—NHS. Also the ε-NH₂ group of all lysines is in this way derivatized in addition to the amino-terminus of the peptide.

Still other labeling methods are, for example, acetic anhydride which can be used to acetylate hydroxyl groups and trimethylchlorosilane, which can be used for less specific labeling of functional groups including hydroxyl groups and amines.

In yet another approach, the primary amino acids are labeled with chemical groups allowing differentiation between the heavy and the light reference peptides by five amu, by six amu, by seven amu, by eight amu or even by larger mass difference. Alternatively, an isotopic labeling is carried out at the carboxy-terminal end of the reference peptides, allowing the differentiation between the heavy and light reference peptides by more than five amu, six amu, seven amu, eight amu or even larger mass differences. Thus, in a preferred embodiment, the quantitative analysis of at least one protein in one sample comprising proteins comprises the steps of: a) preparing a protein peptide mixture wherein the peptides carry an uncommon isotope (e.g., a heavy isotope); b) adding to the protein peptide mixture a known amount of a peptide combo, consisting of a set of synthetic reference peptides, carrying natural isotopes (e.g., a light isotope); c) the protein peptide mixture, also containing the peptide combo, is separated in fractions via a primary chromatographic separation; d) chemical and/or enzymatic alteration of at least the reference peptides and its synthetic peptide combo counterpart; e) isolation of the altered reference peptides and the altered synthetic reference peptides via a secondary chromatographic separation; f) determination by mass spectrometry of the ratio between the peaks heights of the reference peptides versus the synthetic reference peptides and g) calculation of the amount of protein, represented by the reference peptides, in the sample comprising proteins.

In another preferred embodiment, the reversed COFRADIC technology is applied and the isolated reference peptides are unaltered peptides. The above method can equally well be applied to this approach, but in step d) the reference peptides and the peptide combo (the synthetic reference peptides) will remain unaltered and in step e) the unaltered peptides (including the reference peptides and its peptide combo) are isolated.

An example of the reversed COFRADIC technology approach is the isolation of amino-terminal reference peptides of proteins present in a sample. This isolation is designated herein the N-teromics approach.

Thus, in a specific embodiment, the invention provides a method to isolate the amino-terminal reference peptides of the target proteins in a sample comprising proteins. This method comprises the steps of: (1) the conversion of the protein lysine ε-NH₂-groups into guanidyl groups or other moieties, (2) the conversion of the free α-amino-groups at the amino terminal side of each protein, yielding a blocked (not further reactive) group, (3) adding a peptide combo to the sample, (4) digestion of the resulting protein sample yielding peptides with newly generated free NH₂-groups, (5) fractionation of the protein peptide mixture in a primary run, (6) altering the free NH₂-groups of the peptides in each fraction with a hydrophobic, hydrophilic or charged component and (7) isolating the non-altered reference peptides in a secondary run. This approach makes it possible to specifically isolate the amino terminal reference peptides of the proteins in the protein sample, comprising both those amino terminal peptides with a free and those with a blocked α-amino acid group. An application of the latter embodiment is the study of internal proteolytic processing of proteins in a sample comprising proteins

The isolation of a subset of altered reference peptides requires that only a subpopulation of peptides is altered in the protein peptide mixture. In several applications the alteration can be directly performed on the peptides. However, (a) pretreatments of the proteins in the sample and/or (b) pretreatments of the peptides in the protein peptide mixture allow to broaden the spectrum of classes of peptides which can be isolated with the invention. This principle is fully illustrated in WO02077016 which is herein incorporated by reference.

In another preferred embodiment, the quantitative determination of at least one protein in one single sample, comprises the steps of: a) the digestion with trypsin of the protein mixture in H₂¹⁸O into peptides; b) the addition to the resulting protein peptide mixture of a known amount of at least one synthetic reference peptide carrying natural isotopes; c) the fractionation of the protein peptide mixture in a primary chromatographic separation; d) the chemical and/or enzymatic alteration of each fraction on one or more specific amino acids (both the peptides from the protein peptide mixture and the synthetic reference peptides containing the specific amino acid will be altered); e) the isolation of the altered peptides via a second chromatographic separation (these altered peptides comprise both the biological reference peptide and their synthetic reference peptide counterparts); f) the mass spectrometric analysis of the altered peptides and the determination of the relative amounts of the reference peptide and its synthetic reference peptide counterpart. Again, a similar approach can be followed with reference peptides which are simultaneously unaltered peptides.

Also, the above methods can equally be applied in a mode whereby a reference peptide is labeled with the natural isotope and its synthetic reference peptide counterpart is labeled with an uncommon isotope.

Identification of the Peptide Combo and its Corresponding Target Proteins

Peptide combos (consisting of a collection of synthetic reference peptides) are characterized according to their mass-to-charge ratio (m/z) and preferably, also according to their retention time on a chromatographic column (e.g., such as an HPLC column). Synthetic reference peptides are selected which co-elute with reference peptides of identical sequence but which are not labeled. A synthetic reference peptide comprises an amino acid that can be altered such that the altered reference peptide can be isolated with the COFRADIC technology, alternatively in the reverse COFRADIC technology the reference peptides are not altered and are isolated unaltered (e.g., amino-terminal peptides). The reference peptide can be analyzed by fragmenting the peptide. Fragmentation can be achieved by inducing ion/molecule collisions by a process known as collision-induced dissociation (CID) (also known as collision-activated dissociation (CAD). Collision-induced dissociation is accomplished by selecting a peptide ion of interest with a mass analyzer and introducing that ion into a collision cell. The selected ion then collides with a collision gas (typically, argon or helium) resulting in fragmentation.

Generally, any method that is capable of fragmenting a peptide is encompassed within the scope of the present invention. In addition to CID, other fragmentation methods include, but are not limited to, surface induced dissociation (SID) (James and Wilkins, Anal. Chem. 62:1295-1299, 1990; and Williams, et al., Jaser. Soc. Mass Spectrom. 1:413-416, 1990), blackbody infrared radiative dissociation (BIRD); electron capture dissociation (ECD) (Zubarev, et al., J. Am. Chem. Soc. 120:3265-3266, 1998); post-source decay (PSD), LID, and the like. The fragments are then analyzed to obtain a fragment ion spectrum. One suitable way to do this is by CID in multistage mass spectrometry (MSⁿ).

In some occasions, a reference peptide is analyzed by more than one stage of mass spectrometry to determine the fragmentation pattern of the reference peptide and to identify a peptide fragmentation signature. More preferably, a peptide signature is obtained in which peptide fragments have significant differences in m/z ratios to enable peaks corresponding to each fragment to be well separated. Still more preferably, signatures are unique, i.e., diagnostic of a particular reference peptide being identified and comprising minimal overlap with fragmentation patterns of peptides with different amino acid sequences. If a suitable fragment signature is not obtained at the first stage, additional stages of mass spectrometry are performed until a unique signature is obtained. Fragment ions in the MS/MS and MS³ spectra are generally highly specific and diagnostic for peptides of interest.

Multiple reference peptides of a single protein may be synthesized, labeled, and fragmented to identify optimal fragmentation signatures. However, in one aspect, at least two different reference peptides are used as internal standards to identify/quantify a single protein, providing an internal redundancy to any quantitation system. Thus, in a preferred approach peptide analysis of altered or unaltered reference peptides is performed with a mass spectrometer. However, altered or unaltered reference peptides can also be further analyzed and identified using other methods, such as electrophoresis, activity measurement in assays, analysis with specific antibodies, Edman sequencing, etc.

An analysis or identification step can be carried out in different ways. In one way, altered or unaltered reference peptides eluting from the chromatographic columns are directly directed to the analyzer. In an alternative approach, altered or unaltered reference peptides are collected in fractions. Such fractions may or may not be manipulated before going into further analysis or identification. An example of such manipulation consists out of a concentration step, followed by spotting each concentrate on, for instance, a MALDI-target for further analysis and identification.

In a preferred embodiment, altered or unaltered reference peptides are analyzed with high-throughput mass spectrometric techniques. The information obtained is the mass of the altered or unaltered reference peptides. When the peptide mass is very accurately defined, such as with a Fourrier transform mass spectrometer (FTMS), using an internal calibration procedure (O'Connor and Costello, 2000), it is possible to unambiguously correlate the peptide mass with the mass of a corresponding peptide in peptide mass databases and as such identify the altered or unaltered reference peptide. The accuracy of some conventional mass spectrometers is however not sufficient to unambiguously correlate the spectrometrically determined mass of each peptide with its corresponding peptide and protein in sequence databases. To increase the number of peptides that can nevertheless be unambiguously identified, data about the mass of the peptide are complemented with other information.

In one embodiment, the peptide mass as determined with the mass spectrometer is supplemented with the proven knowledge (for instance, proven via neutral loss of 64 amus in the case of methionine sulfoxide altered peptides) that each altered peptide contains one or more residues of the altered amino acid and/or with the knowledge that the peptide was generated following digestion of a sample comprising proteins using a cleavage protease with known specificity. For example, trypsin has the well known property of cleaving precisely at the sites of lysine and arginine, yielding peptides which typically have a molecular weight of between about 500 to 5,000 dalton and having C-terminal lysine or arginine amino acids. This combined information is used to screen databases containing information regarding the mass, the sequence and/or the identity of peptides and to identify the corresponding peptide and protein.

In another embodiment, the method of determining the identity of the parent protein by only accurately measuring the peptide mass of at least one altered or unaltered reference peptide can be improved by further enriching the information content of the selected altered or unaltered reference peptides. As a non-limiting example of how information can be added to the altered or unaltered reference peptides, the free NH₂-groups of these peptides can be specifically chemically changed in a chemical reaction by the addition of two different isotopically labeled groups. As a result of this change, the peptides acquire a predetermined number of labeled groups. Since the change agent is a mixture of two chemically identical but isotopically different agents, the altered or unaltered reference peptides are revealed as peptide twins in the mass spectra.

The extent of mass shift between these peptide doublets is indicative for the number of free amino groups present in the peptide. To illustrate this further, for example, the information content of altered peptides can be enriched by specifically changing free NH₂-groups in the peptides using an equimolar mixture of acetic acid N-hydroxysuccinimide ester and trideuteroacetic acid N-hydroxysuccinimide ester. As the result of this conversion reaction, peptides acquire a predetermined number of CH₃—CO (CD₃—CO) groups, which can be easily deduced from the extent of the observed mass shift in the peptide doublets. As such, a shift of three amus corresponds with one NH₂-group, a three and six amus shift corresponds with two NH₂-groups and a shift of three, six and nine amus reveals the presence of three NH₂-groups in the peptide.

This information further supplements the data regarding the peptide mass, the knowledge about the presence of one or more residues of the altered amino acid and/or the knowledge that the peptide was generated with a protease with known specificity. A yet further piece of information that can be used to identify altered or unaltered reference peptides is the Grand Average of hydrophaticity (GRAVY) of the peptides, reflected in the elution times during chromatography. Two or more peptides, with identical masses or with masses that fall within the error range of the mass measurements, can be distinguished by comparing their experimentally determined GRAVY with the in silico predicted GRAVY.

Any mass spectrometer may be used to analyze the altered or unaltered reference peptides. Non-limiting examples of mass spectrometers include the matrix-assisted laser desorption/ionization (“MALDI”) time-of-flight (“TOF”) mass spectrometer MS or MALDI-TOF-MS, available from PerSeptive Biosystems, Framingham, Mass.; the Ettan MALDI-TOF from AP Biotech and the Reflex III from Brucker-Daltonias, Bremen, Germany for use in post-source decay analysis; the Electrospray Ionization (ESI) ion trap mass spectrometer, available from Finnigan MAT, San Jose, Calif.; the ESI quadrupole mass spectrometer, available from Finnigan MAT or the GSTAR Pulsar Hybrid LC/MS/MS system of Applied Biosystems Group, Foster City, Calif. and a Fourrier transform mass spectrometer (FTMS) using an internal calibration procedure (O'Connor and Costello, 2000).

Protein identification software used in the present invention to compare the experimental mass spectra of the reference peptides with a database of the peptide masses and the corresponding proteins are available in the art. One such algorithm, ProFound, uses a Bayesian algorithm to search protein or DNA database to identify the optimum match between the experimental data and the protein in the database. ProFound may be accessed on the World-Wide Web at http://prowl.rockefeller.edu and http://www.proteometrics.com. Profound accesses the non-redundant database (NR). Peptide Search can be accessed at the EMBL website. See also, Chaurand P. et al. (1999) J. Am. Soc. Mass. Spectrom 10, 91, Patterson S. D., (2000), Am. Physiol. Soc., 59-65, Yates J R (1998) Electrophoresis, 19, 893). MS/MS spectra may also be analyzed by MASCOT (available at http://www.matrixscience.com, Matrix Science Ltd. London).

In another preferred embodiment, isolated altered or unaltered reference peptides are individually subjected to fragmentation in the mass spectrometer. In this way information about the mass of the peptide is further complemented with (partial) sequence data about the altered or unaltered reference peptide. Comparing this combined information with information in peptide mass and peptide and protein sequence databases allows identification of the altered or unaltered reference peptides.

In one approach fragmentation of the altered or unaltered reference peptides is most conveniently done by collision induced dissociation (CID) and is generally referred to as MS² or tandem mass spectrometry. Alternatively, altered peptide ions or unaltered peptide ions can decay during their flight after being volatilized and ionized in a MALDI-TOF-MS. This process is called post-source-decay (PSD). In one such mass spectrometric approach, selected altered or unaltered reference peptides are transferred directly or indirectly into the ion source of an electrospray mass spectrometer and then further fragmented in the MS/MS mode. Thus, in one aspect, partial sequence information of the altered or unaltered reference peptides is collected from the MSⁿ fragmentation spectra (where it is understood that n is larger or equal to 2) and used for peptide identification in sequence databases described herein.

In a particular embodiment, additional sequence information can be obtained in MALDI-PSD analysis when the alfa-NH₂-terminus of the reference peptides is altered with a sulfonic acid moiety group. Altered peptides carrying an NH₂-terminal sulfonic acid group are induced to particular fragmentation patterns when detected in the MALDI-TOF-MS mode. The latter allows a very fast and easy deduction of the amino acid sequence. The ratios of the peak intensities of the heavy and the light peak in each pair of reference peptides (being the synthetic and biological reference peptide) can be measured with mass spectrometry. These ratios give a measure of the relative amount (differential occurrence) of that reference peptide (and its corresponding protein) in each sample. The peak intensities can be calculated in a conventional manner (e.g., by calculating the peak height or peak surface). If a target protein is missing in a sample but not in another, the isolated altered or unaltered peptide (corresponding with this protein) will be detected as one peak which can either contain the heavy or light isotope.

Computer Systems and Databases

The invention also provides methods for generating a database comprising data files for storing information relating to, for example, peptide masses of amino-terminal reference peptides, peptide masses of carboxy-terminal reference peptides and/or internal reference peptides and masses and/or fragmentation signatures for the reference peptides. Preferably, data in the databases also include quantitative values corresponding with the level of proteins (corresponding with the used peptide combo) that is associated or found in a particular cell state (in other words quantitative values which are diagnostic for a cell state e.g., such as a state which is characteristic of a disease, a normal physiological response, a developmental process, exposure to a therapeutic agent, exposure to a toxic agent or a potentially toxic agent, and/or exposure to a condition). Data in the databases also, preferably, include the GRAVY values of the reference peptides. Thus, in one aspect, for a cell state determined by the quantitative expression of at least one protein, a data file corresponding to the cell state will minimally comprise data relating to the mass spectra observed after peptide fragmentation of a reference peptide diagnostic of the protein. Preferably, the data file will include values corresponding to the level of particular proteins present in a cell or tissue. For example, it is known that in a tumor tissue oncogenes are is commonly over-expressed and, thus, the data file will comprise mass spectral data observed after fragmentation of a labeled reference peptide corresponding to a subsequence of a particular oncogene. Preferably, the data file also comprises a value relating to the level of a particular oncogene in a tumor cell. The value may be expressed as a relative value (e.g., a ratio of the level of a particular oncogene in the tumor cell to the level of the oncogene in a normal cell) or as an absolute value (e.g., expressed in nM or as a % of total cellular proteins).

In another aspect, the database also comprises data relating to the source of a cell or tissue or sample which is being evaluated. For example, the database comprises data relating to identifying characteristics of a patient from whom the tissue, sample or body fluid is derived.

The invention further provides a computer memory comprising data files for storing information relating to the diagnostic fragmentation signatures of the peptide combos. Preferably, the database includes data relating to a plurality of cell state profiles, i.e., data relating to the levels of target proteins identified by the peptide combo in a plurality of cells having different cell states or data relating to different time points. For example, profiles of disease states may be included in the database and these profiles will include measurements of levels of one or more proteins, or modified forms thereof, characteristic of the disease state. Profiles of cells exposed to different compounds include measurements of levels of proteins or modified forms thereof characteristic of the response(s) of the cells to the compounds.

In one aspect, the measurements are obtained by performing any of the methods described above. Preferably, the database is in electronic form and the cell state profiles, which are also in electronic form, provide measurements of levels of a plurality of proteins in a cell or cells of one or more subjects. In another aspect, the measurements also include data regarding the site of protein modifications in one or more proteins in a cell. In one preferred aspect, cell state profiles comprise quantitative data relating to target proteins and/or modified forms thereof obtained by using one or more of the methods described above. A variety of data storage structures are available for creating a computer readable medium or memory comprising data files of the database.

The choice of the data storage structure will generally be based on the means chosen to access the stored information. For example, the data can be stored in a word processing text file, formatted in commercially-available software, such as WordPerfect and Microsoft Word, or represented in the form of an ASCII file, stored in a database application, such as DB2, Sybase, Oracle, or the like. The skilled artisan can readily adapt any number of data processor structuring formats (e.g., text files, .pdf files, or database structures) in order to obtain computer readable medium or a memory having recorded thereon data relating to diagnostic fragmentation signatures, e.g., such as mass spectral data obtained after fragmentation of the peptide combo and protein levels.

Correlations between a particular diagnostic signature observed and a cell state (e.g., a disease, genotype, tissue type, etc.) may be known or may be identified using the database described above and suitable statistical programs, expert systems, and/or data mining systems, as are known in the art. In another aspect, the invention provides a computer system comprising databases described herein. In one preferred aspect, the computer system further comprises a user interface allowing a user to selectively view information relating to diagnostic peptide combo values and to obtain information about a cell or tissue state. The interface may comprise links allowing a user to access different portions of the database by selecting the links (e.g., by moving a cursor to the link and clicking a mouse or by using a keystroke on a keypad). The interface may additionally display fields for entering information relating to a sample being evaluated. The system may also be used to collect and categorize peptide fragmentation signatures for different types of cell states to identify reference peptides characteristic of particular cell states. In this aspect, preferably, the system comprises a relational database. More preferably, the system further comprises an expert system for identifying sets of reference peptides that are diagnostic of different cell states. In one aspect, the system is capable of clustering related information. Suitable clustering programs are known in the art and are described in, for example, U.S. Pat. No. 6,303,297.

The system preferably comprises a means for linking a database comprising data files of diagnostic masses and/or fragmentation signatures of peptide combos to other databases, e.g., such as genomic databases, pharmacological databases, patient databases, proteomic databases, and the like. Preferably, the system comprises in combination, a data entry means, a display means (e.g., graphic user interface); a programmable central processing unit; and a data storage means comprising the data files and information described above, electronically stored in a relational database. Preferably, the central processing unit comprises an operating system for managing a computer and its network interconnections. This operating system can be, for example, of the Microsoft Windows family, such as Windows 95, Windows 98, Windows NT, or Windows XP or any new Windows programmed developed. A software component representing common languages may be provided. Preferred languages include C/C++, and JAVAS. In one aspect, methods of this invention are programmed in software packages which allow symbolic entry of equations, high-level specification of processing, and statistical evaluations.

Kits Comprising Peptide Combos

One skilled in the art will readily recognize that the method described in this invention has many advantages. It can be readily modified for automated detection and quantification of target proteins. In one embodiment of the present invention, a machine is provided for processing the sample, cleaving the proteins, sorting the protein targets, and transferring the peptides to mass spectrometry for detection and quantification of the peptide masses, and a computer means for recording and outputting the results of the MS spectra.

Another embodiment is a kit for the detection of a specific target protein in specific sample types, which provides the user with reagents that have been customized for a particular target protein. Thus, in preferred embodiments, the kit contains extraction buffer(s), reagents for a specific alteration of a particular amino acid, protease(s), synthetic reference peptide(s), and precise instructions on their use.

The invention further provides reagents useful for performing the methods described herein. In one aspect, a reagent according to the invention comprises a peptide combo. In one aspect, the peptide combo is labeled with a stable isotope. The invention additionally provides kits comprising one or more synthetic reference peptides labeled with a stable isotope or reagents suitable for performing such labeling.

In certain preferred embodiments, the method utilizes isotopes of hydrogen, nitrogen, oxygen, carbon, or sulfur. Suitable isotopes include, but are not limited to, ²H, ¹³C, ¹⁵N, ¹⁷O, ¹⁸O, or ³⁴S. In another aspect, pairs of reference peptides are provided, comprising identical peptide portions but distinguishable labels, e.g., peptides may be labeled at multiple sites to provide different heavy forms of the peptide. Pairs of reference peptides corresponding to modified and unmodified peptides also can be provided.

In one aspect, a kit comprises reference peptides comprising different peptide sub-sequences from a single known protein. In another aspect, the kit comprises reference peptides corresponding to different known or predicted modified forms of a polypeptide. In a further aspect, the kit comprises a peptide combo corresponding to a family of proteins, e.g., such as proteins involved in a molecular pathway (a signal transduction pathway, a cell cycle, a hedgehog pathway, a proteolysis pathway etc.), which are diagnostic of particular disease states, developmental stages, tissue types, genotypes, etc. The synthetic reference peptides from a peptide combo may be provided in separate containers or as a mixture or “cocktail” of synthetic reference peptides. In one aspect, a peptide combo consists of a plurality of synthetic reference peptides, e.g., representing a MAPK signal transduction pathway. Preferably, the kit comprises a peptide combo comprising at least two, at least about five, at least about ten or more, of synthetic reference peptides corresponding to any of, for example, MAPK, GRB2, mSOS, ras, raf, MEK, p85, KHS1, GCK1, HPK1, MEKK 1-5, ELK1, c-JUN, ATF-2, MLK1-4, PAK, MKK, p38, a SAPK subunit, hsp27, and one or more inflammatory cytokines.

In another aspect, a peptide combo is provided which comprises at least about two, at least about five or more, of synthetic reference peptides which correspond to proteins selected from the group including, but not limited to, PLC iso-enzymes, phosphatidyl-inositol 3-kinase (PI-3 kinase), an actin-binding protein, a phospholipase D isoform, (PLD), and receptor and non-receptor PTKs. In another aspect, a peptide combo is provided which comprises at least about two, at least about five, or more, of synthetic reference peptides which correspond to proteins involved in a JAK signaling pathway, e.g., such as one or more of JAK 1-3, a STAT protein, IL-2, TYK2, CD4, IL-4, CD45, a type I interferon (IFN) receptor complex protein, an IFN subunit, and the like.

In a further aspect, a peptide combo is provided which comprises at least about two, at least about five, or more of peptide internal standards which correspond to cytokines. Preferably, such a set comprises standards selected from the group including, but not limited to, pro- and anti-inflammatory cytokines (which may each comprise their own set or which may be provided as a mixed set of synthetic reference peptides).

In still another aspect, a peptide combo is provided which comprises a peptide diagnostic of a cellular differentiation antigen. Such kits are useful for tissue typing. In one aspect, a combo peptide corresponding to known variants or mutations in a target polypeptide, or which are randomly varied to identify all possible mutations in an amino acid sequence, can also be provided in a kit.

In another aspect, a combo peptide corresponding to proteins expressed from nucleic acids comprising single nucleotide polymorphisms can be provided. Such combo peptides may include synthetic reference peptides corresponding to variant proteins selected from the group comprising BRCA1, BRCA2, CFTR, p53, a JAK protein, a STAT protein, blood group antigens, HLA proteins, MHC proteins, G-Protein Coupled Receptors, apolipoprotein E, kinases (e.g., such as hCdsl, MTKs, PTK, CDKs, STKs, CaMs, and the like), phosphatases, human drug metabolizing proteins, viral proteins, including but not limited to viral envelope proteins (e.g., an HIV envelope protein), transporter proteins and the like.

In one aspect, a synthetic reference peptide comprises a label associated with a modified amino acid residue, such as a phosphorylated amino acid residue, a glycosylated amino acid residue, an acetylated amino acid residue, a farnesylated residue, a ribosylated residue, and the like.

In another aspect, a pair of reagents is provided, a synthetic reference peptide corresponding to a modified peptide and a reference peptide corresponding to a peptide, identical in sequence but not modified.

In another aspect, one or more control synthetic reference peptide internal standards can be provided. For example, a positive control may be a synthetic reference peptide internal standard corresponding to a constitutively expressed protein, while a negative synthetic reference peptide internal standard may be provided corresponding to a protein known not to be expressed in a particular cell or species being evaluated.

In still another aspect, a kit comprises a labeled reference peptide internal standard as described above and software for analyzing mass spectra (e.g., such as SEQUEST and other software herein described). Preferably, the kit also comprises a means for providing access to a computer memory comprising data files storing information relating to the masses and/or diagnostic fragmentation signatures of one or more reference peptide(s) or reference peptide(s) internal standard(s). Access may be in the form of a computer readable program product comprising the memory, or in the form of a URL and/or password for accessing an internet site for connecting a user to such a memory.

In another aspect, the kit comprises diagnostic fragmentation signatures (e.g., such as mass spectral data) in electronic or written form, and/or comprises data, in electronic or written form, relating to amounts of target proteins characteristic of one or more different cell states and corresponding to reference peptides which produce the fragmentation signatures. The kit may further comprise expression analysis software on computer readable medium, which is capable of being encoded in a memory of a computer having a processor and capable of causing the processor to perform a method comprising: determining a test cell state profile from reference peptide masses and/or reference peptide fragmentation patterns in a test sample comprising a cell with an unknown cell state or a cell state being verified; receiving a diagnostic profile characteristic of a known cell state; and comparing the test cell state profile with the diagnostic profile.

In one aspect, the test cell state profile comprises values of levels of reference peptides in a test sample that correspond to one or more reference peptide internal standards provided in the kit. The diagnostic profile comprises measured levels of the one or more peptides in a sample having the known cell state (e.g., a cell state corresponding to a normal physiological response or to an abnormal physiological response, such as a disease). Preferably, the software enables a processor to receive a plurality of diagnostic profiles and to select a diagnostic profile that most closely resembles or “matches” the profile obtained for the test cell state profile by matching values of levels of proteins determined in the test sample to values in a diagnostic profile, to identify substantially all of a diagnostic profile which matches the test cell state profile. Substantially all of a diagnostic profile is matched by a test cell state profile when most of the cellular constituents (e.g., proteins in the proteome) which are diagnostic of the cell state, are found to have substantially the same value in the two profiles within a margin provided by experimental error. Preferably, at least about 75% of the target proteins can be matched, at least about 80%, at least about 85%, at least about 90% or at least about 95% can be matched. Preferably, where one, or only a few proteins (e.g., less than ten) are used to establish a diagnostic profile, preferably all of the proteins have substantially the same value.

Variations, modifications, and other implementations of what is described herein will occur to those of ordinary skill in the art without departing from the spirit and scope of the invention as described and claimed herein and such variations, modifications, and implementations are encompassed within the scope of the invention. All of the references identified hereinabove are expressly incorporated herein by reference. The methods, instruments and procedures described herein can be used for a variety of purposes. Because of the sensitivity and specificity of the analysis one skilled in the art will readily recognize uses for this methodology. What follows is a representative list of uses in specific areas where a current need exists for a quick and reliable analysis.

Uses of Peptide Combos

The methods provided in the present invention to quantify at least one protein in a sample comprising proteins can be broadly applied to quantify proteins of different interest. For example, diagnostic or prognostic assays can be developed by which the level of one or more proteins is determined in a sample by making use of the present invention.

In one embodiment, a combo peptide can be used to quantify specific known splice variants of one or more particular proteins in a sample. If a particular splice variant is known from a specific protein and the splice variant is aimed to be detected then a synthetic reference peptide can be synthesized that only corresponds with the splice variant of a particular protein. Indeed, it often happens that due to exon skipping new junctions are formed and as such a specific reference peptide can be chosen that not occurs in the parent protein and only occurs in the splice variant. However, in many cases it is advised to choose two or more reference peptides in order to distinguish between the parent protein and the splice variant of interest. Also it is common that a particular splice variant is expressed together with the parent protein in the same cell or tissue and, thus, both are present in the sample. Often the expression levels of the particular splice variant and the parent protein are different. The detection and the abundance between the reference peptides can be used to calculate the expression levels between the splice variant and its parent protein.

In yet another embodiment, it is well known that drugs can highly influence the expression of particular proteins in a cell. With the current method it is possible to accurately measure the amount of one or a set of proteins of interest under different experimental conditions. As such, equivalent technologies, such as genomic applications, can be applied on the protein level comprising pharmacoproteomics and toxicoproteomics. Though gene markers of disease have received significant attention with the sequencing of the human genome, protein markers are more useful in many situations. For example, a diagnostic assay based on a combo peptide representing protein disease markers can be developed basically for any disease of interest. Most conveniently such disease markers can be quantified in cell, tissue or organ samples or body fluids comprising, for instance, blood cells, plasma, serum, urine, sperm, saliva, sputum, peritoneal lavage fluid, feces, tears, nipple aspiration fluid, synovial fluid or cerebrospinal fluid.

Reference peptides for protein disease markers can then, according to the present invention, for example, be used for monitoring if the patient is a fast or slow disease progressor, if a patient is likely to develop a certain disease and even to monitor the efficacy of treatment. Indeed, in contrast to genetic markers, such as SNPs, levels of protein disease markers, indicative for a specific disease, could change rapidly in response to disease modulation or progression. Reference peptides for protein disease markers can, for instance, also be used, according to the present invention, for an improved diagnosis of complex genetic diseases, such as, for example, cancer, obesity, diabetes, asthma and inflammation, neuropsychiatric disorders, including depression, mania, panic disorder and schizophrenia. Many of these disorders occur due to complex events that are reflected in multiple cellular and biochemical pathways and events. Therefore, many proteins markers may be found to be correlated with these diseases.

The present invention allows quantification of one to several hundreds of protein disease markers simultaneously. Also the absolute quantification of protein markers, using the current invention, could lead to a more accurate diagnostic sub-classification.

In another specific embodiment, synthetic reference peptides representing modified and unmodified forms of a protein can be used together, to determine the extent of protein modification in a particular sample of proteins, i.e., to determine what fraction of the total amount of protein is represented by the modified form. Preferably, the label in the synthetic reference peptide is attached to a peptide comprising a modified amino acid residue or to an amino acid residue that is predicted to be modified in a target polypeptide.

In one aspect, multiple reference peptides representing different modified forms of a single protein and/or peptides representing different modified regions of the protein are added to a sample and corresponding target peptides (bearing the same modifications) are detected and/or quantified. Preferably, a peptide combo representing both modified and unmodified forms of a protein are provided in order to compare the amount of modified protein observed to the total amount of protein in a sample.

In another embodiment, reference peptides are synthesized which correspond to a single amino acid subsequence of a target polypeptide but which vary in one or more amino acids. Such a peptide combo may correspond to known variants or mutations in the target polypeptide or can be randomly varied to identify all possible mutations in an amino acid sequence.

In one preferred aspect, a peptide combo corresponding to proteins expressed from nucleic acids comprising single nucleotide polymorphisms are synthesized to identify variant proteins encoded by such nucleic acids. Thus, reference peptides can be generated corresponding to SNPs which map to coding regions of genes and can be used to identify and quantify variant protein sequences on an individual or population level. SNP sequences can be accessed through the Human SNP database available at http://www-genome.wi.mit.edu/SNP/human/index.html. Synthetic reference peptides may also be used to scan for mutations in proteins including, but not limited to, BRCA1, BRCA2, CFTR, p53, blood group antigens, HLA proteins, MHC proteins, G-Protein Coupled Receptors, apolipoprotein E, kinases (e.g., such as hCdsl, MTKs, PTK, CDKs, STKs, CaMs, and the like), phosphatases, human drug metabolizing proteins, viral proteins, such as a viral envelope proteins (e.g., HIV envelope proteins), transporter proteins, and the like.

In a further aspect, synthetic reference peptides corresponding to different modified forms of a protein are synthesized, providing internal standards to detect and/or quantitate changes in protein modifications in different cell states.

In still a further aspect, synthetic reference peptides are generated which correspond to different proteins in a molecular pathway and/or modified forms of such proteins (e.g., proteins in a signal transduction pathway, cell cycle, hedgehog pathway, metabolic pathway, blood clotting pathway, etc.) providing panels of internal standards to evaluate the regulated expression of proteins and/or the activity of proteins in a particular pathway.

In one aspect, a known amount of a labeled reference peptide corresponding to a target protein to be detected and/or quantitated, is added to a sample, such as a cell lysate. For example, an amount of about 10 picomoles, 5 picomoles, 1 picomole, 500 femtomoles, 100 femtomoles, 10 femtomoles or less of a reference peptide is spiked into the sample.

In still another aspect, a peptide combo is added to a sample that represents different proteins in a molecular pathway (e.g., a signal transduction pathway, a cell cycle, a metabolic pathway, a blood clotting pathway) and/or different modified forms of such proteins. In this aspect, the function of the pathway is evaluated by monitoring the presence, absence or quantity of particular pathway proteins and/or their modified forms. Multiple pathways may be evaluated at a time and/or at different time points by combining mixtures of different pathway peptide combos.

In a further aspect, a peptide combo represent proteins and/or modified forms thereof whose presence is diagnostic of a particular tissue type (e.g., neural proteins, cardiac proteins, skin proteins, lung proteins, liver proteins, pancreatic proteins, kidney proteins, proteins characteristic of reproductive organs, etc.). These can be used separately or in combination to perform tissue-typing analysis. Synthetic reference peptides may represent proteins or modified forms thereof whose presence is characteristic of a particular genotype (e.g., such as HLA proteins, blood group proteins, proteins characteristic of a particular pedigree, etc.). These can be used separately or in combination to perform forensic analyses, for example.

In still another embodiment, synthetic reference peptides are used in prenatal testing to detect the presence of a congenital disease or to quantitate protein levels diagnostic of a chromosomal abnormality. Synthetic reference peptides may represent proteins or modified forms thereof whose presence is characteristic of particular diseases. Such reference peptides may correspond to target proteins diagnostic of neurological disease (e.g., neurodegenerative diseases, including, but not limited to, Alzheimer's disease; amyotrophic lateral sclerosis; dementia, depression; Down's syndrome; Huntington's disease; peripheral neuropathy; multiple sclerosis; neurofibromatosis; Parkinson's disease; and schizophrenia). These standards can be used separately or in combination to diagnose a neurological disease. Preferably, sets of peptide combos are used so that diagnostic fragmentation signatures can be evaluated for a number of different diseases in a single assay. Thus, a sample may be obtained from a patient who presents with general symptoms associated with a neurological disease, and a combo peptide comprising reference peptides for proteins diagnostic of different neurological diseases can be added to the sample. The peptide combo may include a reference peptide corresponding to a control target protein, such as a constitutively expressed protein of known abundance. A negative standard (e.g., such as a reference peptide corresponding to a plant protein—when a mammalian system is used) may also be provided.

Similarly, peptide combos can be used to diagnose immune diseases, including, but not limited to, acquired immunodeficiency syndrome (AIDS); Addison's disease; adult respiratory distress syndrome; allergies; ankylosing spondylitis; amyloidosis; anemia; asthma; atherosclerosis; autoimmune hemolytic anemia; autoimmune thyroiditis; bronchitis; cholecystitis; contact dermatitis; Crohn's disease; atopic dermatitis; dermatomyositis; diabetes mellitus; emphysema; episodic lymphopenia with lymphocytotoxins; erythroblastosis fetalis; erythema nodosum; atrophic gastritis; glomerulonephritis; Goodpasture's syndrome; gout; Graves' disease; Hashimoto's thyroiditis; hypereosinophilia; irritable bowel syndrome; myasthenia gravis; myocardial or pericardial inflammation; osteoarthritis; osteoporosis; pancreatitis; and polymyositis. Similarly, peptide combos can be used to characterize infectious diseases, respiratory diseases, reproductive diseases, gastrointestinal diseases, dermatological diseases, hematological diseases, cardiovascular diseases, endocrine diseases, urological diseases, and the like. Because peptide combos provide diagnostic fragmentation signatures for detecting and/or quantitating proteins or modified forms thereof, changes in the presence or amounts of such fragmentation signatures in a sample of proteins from a cell (e.g., such as a cell lystate), as discussed above, can be diagnostic of a cell state.

In a particular embodiment, changes in cell state are evaluated after exposure of the cell to a compound. Compounds are selected which are capable of normalizing a cell state, e.g., by selecting for compounds which alter the quantification levels of a set of target proteins from those characteristic of abnormal physiological responses to those representative of a normal cell. For example, a three way comparison of healthy, diseased, and treated diseased individuals can identify which compounds are able to restore a disease cell state to a one that more closely resembles a normal cell state. This can be used to screen for drugs or other therapeutic agents, to monitor the efficacy of treatment, and to detect or predict the occurrence of side effects, whether in a clinical trial or in routine treatment, and to identify protein targets which are more important to the manifestation and treatment of a disease. Compounds which can be evaluated include, but are not limited to: drugs; toxins; proteins; polypeptides; peptides; amino acids; antigens; cells, cell nuclei, organelles, portions of cell membranes; viruses; receptors; modulators of receptors (e.g., agonists, antagonists, and the like); enzymes; enzyme modulators (e.g., such as inhibitors, cofactors, and the like); enzyme substrates; hormones; nucleic acids (e.g., such as oligonucleotides; polynucleotides; genes, cDNAs; RNA; antisense molecules, ribozymes, aptamers), and combinations thereof. Compounds also can be obtained from synthetic libraries from drug companies and other commercially available sources known in the art (e.g., including, but not limited, to the LeadQuest library) or can be generated through combinatorial synthesis using methods well known in the art.

In one aspect, a compound is identified as a modulating agent if it alters the site of modification of a polypeptide and/or if it alters the amount of modification by an amount that is significantly different from the amount observed in a control cell (e.g., not treated with compound) (setting p values to<0.05). In another aspect, a compound is identified as a modulating agent, if it alters the amount of the polypeptide (whether modified or not).

Peptide combos can also be used as biomarkers in following biomedical applications: (1) preclinical drug development, (2) development improved animal models, (3) biomarkers related with toxicology, (4) clinical drug development (e.g., patient selection, monitoring drug efficacy, discriminating responders from non-responders), (5) guidance marketed drugs (e.g., selection responders, evaluation drug resistance, post-launch differentiation of competitors), (6) prognostic disease markers, (7) diagnostic disease markers, (8) drug target validation and selection (e.g., simultaneous analysis of the functional state of the Epidermal Growth Factor Receptor (EGF)-family, involved in multiple solid tumors), (9) monitoring protein splicing, (10) drug lead profiling (e.g., lead profiling of inhibitors of gamma-secretase, a key drug target in Alzheimer disease, using synthetic N-terminal peptides; lead profiling of inhibitors of p38MAPK, a kinase involved in inflammatory diseases and chronic obstructive pulmonary disease (COPD), using synthetic phosphopeptides), (11) pathway analysis, (12) answering basic disease biology questions by monitoring post-translational modifications (phosphorylation, acetylation methylation, ubiquitination, . . . ), (13) simultaneous functional and spatial analysis G-protein coupled receptors (GPCRs), belonging to the most important class of drug targets used in pharma and biotech (i.e., protein expression studies in small subregions of the brain, the gastro-intestinal tract, . . . ) and (14) peptide combos also have applications in the fields of food and feed, cosmetics, agriculture and animal breeding (e.g., biomarkers to aid the development and to track the efficacy of nutraceuticals in achieving desired results; biomarker-assisted selection programs to support breeding and marketing of food-producing animals possessing enhanced genetic merit for value (e.g., the study of meat quality changes in transgenic animals produced to improve feed-efficiency, carcass yield, and lean tissue); biomarker assisted safety assessment of cosmetics (toxicokinetics, carcinogenicity, teratogenicity, reproductive toxicity); evaluation of the performance of microbial starter cultures in different food applications (e.g., yogurt); quantification of the occurrence of proteins expressed in corn seeds in different stages of development; quantification of the presence of proteinaceous allergens in food products).

Sputum is an easily obtainable sample source for the early recognition of diseases affecting the airways. While serum and plasma, which are easier to access, may indicate the presence of an already established disease (and, therefore, are useful for prediction of therapy response), sputum may permit detection of much earlier lung lesions. Furthermore, sputum locates the disease to the airways, therefore, they are organ specific and, thus, provide the opportunity to isolate relevant (diseased tissue specific) drug targets or protein therapeutics.

In the event a lung disease biomarker consists of multiple differentially expressed sputum proteins, a Peptide Combo, can be used to screen for such biomarker. A specific Peptide Combo comprises a combined set of smartly selected reference peptides, each reference peptide representing one of the differentially expressed proteins. The addition of a known amount of such Peptide Combo to the biological sample and applying the quantitative COFRADIC strategy then allows determination of the abundance of each of the proteins. The Peptide Combos represents a significant shortcut in biomarker assay development because there is no need to develop antibodies and to generate an immunoassay.

### EXAMPLES

1. A Peptide Combo to Aid Lead Profiling of Gamma-Secretase (γ-Secretase) Inhibitors

Gamma-secretase is one of the major drug targets for Alzheimer disease (AD). While processing of APP via gamma-secretase generates Amyloid beta, the culprit peptide in AD, gamma-secretase is involved in processing many other substrates as well (Haas and Steiner, Trends Cell Biol. 12, 556-562, 2002). This redundancy hampers the development of specific secretase inhibitors. A gamma-secretase Peptide Combo can be designed comprising synthetic reference peptides that are capable of determining the expression level of the known gamma-secretase substrates, both in neuronal and non-neuronal cell types. This gamma-secretase Peptide Combo will contain amino terminal peptides corresponding to the novel amino-termini generated following gamma-secretase cleavage of its substrates. Such a Peptide Combo is a unique tool to profile the specificity of direct and indirect gamma-secretase inhibitors measuring changes in the nature of products resulting from gamma-secretase cleavage. A gamma-secretase Peptide Combo consists of at least one of the amino-terminal synthetic signature peptides for at least one of the proteins presented in Table 1.

The peptides in Table 1 are generated following a partial Arg-C digest and application of the Reverse COFRADIC technology (N-teromics or isolation of amino-terminal peptides). Their mass limit is set between 400 and 5,000 Da.

2. A Peptide Combo Comprising Peptides Corresponding to Different Proteins in a Molecular Pathway, Wherein Each Peptide Comprises a Signature Diagnostic of a Protein in the Molecular Pathway

The Hedgehog (Hh) signaling pathway is involved in both development and human diseases (mainly cancer induction) in a wide range of organisms (Mullor et al., Trends Cell Biology 12, 562-569, 2002). The end point of the Hedgehog signal-transduction cascade is activation of the GLI/Ci zinc-finger transcription factors. Several components of the Hh pathway have been first identified in flies and a number of them are not yet characterized in humans. Hh, an extracellular ligand, is secreted by discrete subsets of cells in many organs. After secretion, Hh molecules form multimeric complexes. Their transport requires EXT1 and EXT2, the human homologs of Tout-velu in Drosophila. Two membrane proteins function to receive the Hh signal: Patched (PTC) and Smoothened (SMO). Hh binding to PTC releases the basal repression of SMO by PTC and SMO then signals intracellularly to transduce the Hh signal to the nucleus. This is performed by regulation of the GLI transcription factors (GLI1, GLI2, GLI3), relying both on GLI activating function and on inhibiting GLI repressor formation. Inside the cell and downstream of SMO, a large number of proteins activate (PKA, COS2, Suppressor of Fused (SUFU) or repress or attenuate the Hh pathway (Fused, Casein kinase-1 and GSK3) via regulation of Gli/Ci processing, activity, and localization.

Alterations in different components of the Hh pathway can lead to different phenotypes, although there is a good degree of consistency, implying the linearity of the pathway. For example, on the one hand, alterations in several loci have been associated with Holoprosencephaly (SHH, PTC and ZIC2). On the other hand, diseases associated with growth regulation, such as basal cell carcinomas, medulloblastomas, rhabdomyosarcomas and Hereditary multiple exostosis (benign bone tumors) can arise from gain of function of SHH, GLI or SMO proteins, or loss of function of PTC, SUFU or EXT proteins.

As the Hh pathway is involved in many developmental events, it will also likely be associated with further human syndromes. Several therapeutic approaches to restore the normal status of Hh signaling might be feasible. Most attractive is the development of drugs that agonize or antagonize different negative or positive components of the Hh pathway. The small molecule cyclopamine, its derivatives or functional analogs could be good therapeutic agents to fight diseases caused by activation of the Hh pathway at the receptor level.

To track protein expression in the entire Hh pathway, independent of cell type, we can make use of the Hh pathway Peptide Combo. Such Peptide Combo consists of at least one of the methionine containing signature peptides, or at least one of the cysteine containing peptides, or at least one of the methionine and cysteine containing peptides for at least one of the proteins presented in Table 2.1-2.3.

These peptides are generated following a Trypsin digest in which one miss-cleavage is allowed and application of the Met-COFRADIC, Cys-COFRADIC or Met+Cys-COFRADIC technology respectively. Their mass limit is set between 600 and 4000 Da. Peptide sets for the 12-transmembrane-domain protein PTC and the 7-transmembrane-domain protein SMO are selected for their position in the non-transmembrane part of the proteins, which is the most accessible for protease cleavage.

3. G-Protein Coupled Receptors (GPCRs)

The superfamily of G-protein Coupled Receptors (GPCRs) is the most successful of any target class in terms of therapeutic benefit and commercial sales. In 2000, 26 of the top 100 pharmaceutical products were compounds that target GPCRs accounting for sales over US$23 billion.

G-protein-coupled receptors (GPCRs) constitute a large family of seven-transmembrane receptors that transmit extracellular signals from bound ligand to intracellular G proteins, which in turn activate or inhibit various intracellular second messenger systems. GPCRs are divided into three broad groups: those with known ligands, which are sorted by subfamily based on ligand (endogenous ligands include neurotransmitters, hormones, and chemotactic factors); sensory receptors, which are involved in sensory pathways (olfactory, pheromone, taste); and orphan receptors, for which ligands have not yet been identified.

These hydrophobic membrane bound proteins also constitute the most difficult drug target class to analyze with 2D-PAGE. Obtaining antibodies against the extracellular domains of GPCRs has proved notoriously difficult as well because of the relative short sequence and the constrained nature of the extracellular loops and, for many receptors, the short nature of the N-terminal domain. Combining GPCR specific reference peptides creates a broadly applicable Peptide Combo which allows profiling of GPCR expression in any given type of cells at all stages of the drug discovery process, without the use of antibodies.

Table 3 contains the signature peptides to compose a Peptide Combo a) to study the GPCRs targeted by the best-selling GPCR therapeutics, b) to study the Secretin-like GPCR family B, and c) to study orphan GPCRs.

3a. GPCR Therapeutic Targets

A GPCR Peptide Combo to study the most successful GPCR targets in terms of therapeutic benefit and commercial sales consists of at least one of the methionine containing signature peptides, or at least one of the cysteine containing peptides, or at least one of the methionine and cysteine containing peptides for at least one of the proteins presented in Table 3a.1-3a.3. These peptides are generated following a Trypsin digest in which one miss-cleavage is allowed and application of the Met-COFRADIC, Cys-COFRADIC or Met+Cys-COFRADIC technology respectively. Their mass limit is set between 600 and 4000 Da. Peptide sets are selected for their position in the non-transmembrane part of the proteins, which is the most accessible for protease cleavage.

3b. GPCR Family B, Secretin-Like.

A GPCR Peptide Combo to study the Secretin-like family B GPCRs consists of at least one of the methionine containing signature peptides, or at least one of the cysteine containing peptides, or at least one of the methionine and cysteine containing peptides for at least one of the proteins presented in Table 3b.1-3b.3. These peptides are generated following a Trypsin digest in which one miss-cleavage is allowed and application of the Met-COFRADIC, Cys-COFRADIC or Met+Cys-COFRADIC technology respectively. Their mass limit is set between 600 and 4000 Da. Peptide sets are selected for their position in the non-transmembrane part of the proteins, which is the most accessible for protease cleavage.

3c. Orphan GPCRs

For many orphan receptors there is currently little information available beyond the gene sequence. Knowledge about cell-specific localization and disease association is essential for the rapid and accurate prioritization of these potential drug targets. While expression can be analyzed at the RNA level, ideally expression should be confirmed at the protein level. Obtaining antibodies directed against the extracellular domains of GPCRs has proved notoriously difficult because of the relatively short sequence and constrained nature of the extracellular loops and, for many receptors, the short nature of the N-terminal domain. As antibodies have so far been required for target validation studies to implicate GPCRs in disease, orphan GPCR Peptide Combos will obviate this need. A GPCR Peptide Combo to study currently orphan GPCRs would consist of at least one of the methionine containing signature peptides, or at least one of the cysteine containing peptides, or at least one of the methionine and cysteine containing peptides for at least one of the proteins presented in Table 3c.1-3c.3. These peptides are generated following a Trypsin digest in which one miss-cleavage is allowed and application of the Met-COFRADIC, Cys-COFRADIC or Met+Cys-COFRADIC technology respectively. Their mass limit is set between 600 and 4000 Da. Peptide sets are selected for their position in the non-transmembrane part of the proteins, which is the most accessible for protease cleavage.

4. A Peptide Combo to Analyze Splicing at the Protein Level

4a. A Peptide Combo to Distinguish COX splice Isoforms

Some of the most widely used medicines today are nonsteroidal anti-inflammatory drugs (NSAIDs). These drugs act on cyclooxygenase (COX) enzymes. Two COX isozymes, COX1 and COX2 catalyze the rate-limiting step of prostaglandin synthesis. Recently, novel isoforms of COX1 were discovered (Chandrasekharan et al., PNAS 99, 13926-13931, 2002). While it is known that COX1 functions in platelet activation, it is only possible to analyze the novel identified COX1 isoforms at the protein level as platelets are anucleate and do not contain DNA. COX isoform-specific Peptide Combos allow to study these COX isoforms, to interrogate NSAIDs method of action and to improve development of novel NSAIDs. A COX splicing Peptide Combo consists of at least one of the methionine containing signature peptides, or at least one of the cysteine containing peptides, or at least one of the methionine and cysteine containing peptides for each of the proteins presented in Table 4a.1-4a.3.

These peptides are generated following a Trypsin digest in which one miss-cleavage is allowed and application of the Met-COFRADIC, Cys-COFRADIC or Met+Cys-COFRADIC technology respectively. Their mass limit is set between 600 and 4000 Da.

4b. A Peptide Combo to Distinguish VEGF-A Splice Isoforms

Vascular endothelial growth factor (VEGF) is a highly specific factor for vascular endothelial cells. Seven VEGF-A isoforms (splice variants 121, 145, 148, 165, 183, 189 and 206) are generated as a result of alternative splicing from a single VEGF-A gene. These differ in their molecular weights and in biological properties, such as their ability to bind to cell-surface heparan sulfate proteoglycans. Deregulated VEGF-A expression contributes to the development of solid tumors by promoting tumor angiogenesis. VEGF-A189 expression, for instance, is related to angiogenesis and prognosis in certain human solid tumors. VEGF-A189 expression is also related to the xenotransplantability of human cancers into immunodeficient mice in vivo.

A VEGF splicing Peptide Combo consists of at least one of the cysteine containing peptides, for each of the VEGF isoforms presented in Table 4b (except the VEGF-A165 and VEGF-A148 isoform).

These peptides are generated following a Trypsin digest in which one miss-cleavage is allowed and application of the Cys-COFRADIC technology. Their mass limit is set between 600 and 4000 D.

1. A process of identifying a peptide combo wherein said peptide combo corresponds with a family of proteins and wherein each of the members of said peptide combo is derived from a unique protein from said family of proteins, said process comprising the steps of:
a) generating peptides by applying a digest on said family of proteins, and
b) identifying a peptide combo with chosen properties.

2. The process of claim 1 wherein generating peptides comprises generating peptides by applying an in silico digest on said family of proteins followed by constructing a relational database comprising said peptides with a predicted mono isotopic weight within the range of 600-4000 Da.

3. The process of claim 1 wherein said family of proteins includes membrane proteins and wherein the peptides generated in step a) have less than 20% coverage in the transmembrane area.

4. The process of claim 3 wherein said membrane proteins are G-protein coupled receptors.

5. The process of claim 1, wherein said chosen properties are the presence of specific amino acids which can be chemically and/or enzymatically altered.

6. The process of claim 5 wherein said specific amino acids are selected from the group consisting of methionine, cysteine, and a combination of methionine and cysteine.

7. The process of claim 1, wherein said chosen property is an amino-terminal peptide.

8. A peptide combo comprising at least two peptides obtainable by the processes of claim 1.

9. The peptide combo of claim 8 wherein said peptides are isotopically labeled.

10. The peptide combo of claim 8 that comprises peptides derived from G-protein coupled receptors.

11. The peptide combo of claim 8 that comprises peptides derived from protease substrates.

12. The peptide combo of claim 11 wherein said protease is gamma secretase.

13. A method of determining the abundance of each protein belonging to a family of proteins, said method comprising the steps of:
(a) adding to a protein or peptide mixture a known amount of the peptide combo of claim 8;
(b) separating said mixture into fractions of peptides via chromatography in a chromatographic column system of a type;
(c) chemically, enzymatically, or chemically and enzymatically, altering at least one amino acid of at least one of the peptides in each fraction of peptides separated via chromatography;
(d) isolating the altered peptides out of each fraction via chromatography, wherein the chromatography is performed with the same type of chromatographic column system as in step (b);
(d) performing mass spectrometric analysis of the altered peptides and detecting twin peaks in said mass spectrometric analysis;
(e) calculating the peak surfaces of each of the twin peaks, thereby obtaining a ratio that corresponds with the amount of the reference peptide in the sample, and
f) determining the identity of said reference peptides and their corresponding proteins.

14. The method according to claim 13 wherein in step c) at least one amino acid is chemically, or enzymatically, or chemically and enzymatically altered in the majority of the peptides in each fraction and wherein in step d) the non-altered peptides are isolated out of each fraction via chromatography.

15. The method according to claim 13 wherein step a) is preceded by one or more pre-treatment steps.

16. The method according to claim 13 wherein the chromatographic conditions of steps a) and c) are the same or substantially similar.

17. The method according to claim 13, wherein determining the identity of the reference peptides is performed by a method selected from the group consisting of a tandem mass spectrometric method, Post-Source Decay analysis, measurement of the mass of the peptides, and measurement of the mass of the amino-terminal peptides, in combination with database searching.

18. The method according to claim 17 wherein the determining the identity of the reference peptides is further based on one or more of the following: (a) the presence of the altered amino acid; (b) the determination of the number of free amino acids in the reference peptides, (c) the knowledge about the cleavage specificity of the protease used to generate the protein peptide mixture, and (d) the grand average of the hydropathicity of the peptides.

19. The method according to claim 13, wherein the protein peptide mixture of step (a) is isotopically labeled and the synthetic reference peptide carries a natural isotope.

20. The method according to claim 13, wherein the samples are biological samples.

21. The method according to claim 20 to diagnose a disease or a pre-disposition to a disease in a subject from whom the biological sample has been taken.

22. A method of quantifying splice variants of one or more target proteins, said method comprising the method according to claim 13 to quantify splice variants of one or more target proteins.

23. A method of predicting a response to therapeutic modulation of a disease, said method comprising using the method of claim 13 to predict response to therapeutic modulation of a disease.


SEQUENCE LISTING (7 sequences)

SEQ ID NO: 1 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 2 - protein, 215 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 3 - protein, 209 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 4 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 5 - protein, 174 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 6 - protein, 171 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 7 - protein, 147 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENCDKPRR